(19) United States
(12) Patent Application Publication
(10) Pub. No.: US 2010/0247686 A1
(43) Pub. Date: Sep. 30, 2010

(54) COMPOSITIONS AND METHODS FOR OBESITY, DIABETES AND METABOLIC SYNDROME CONTROL AND MANAGEMENT

(76) Inventor: Litao Zhong, San Diego, CA (US)

Correspondence Address: MORRISON & FOERSTER LLP, 755 PAGE MILL RD, PALO ALTO, CA, 94304-1018, US

(21) Appl. No.: 12/531,863

(22) PCT Filed: Apr. 4, 2008
(86) PCT No.: PCT/US08/04464

§ 371 Date: May 20, 2010

Related U.S. Application Data
(60) Provisional application No. 60/921,906, filed on Apr. 4, 2007.

(51) Int. Cl. A61K 36/87; A61P 3/10; A61P 3/04

(52) U.S. Cl. 424/729; 424/769

(57) ABSTRACT

The invention relates to compositions and methods for controlling food, energy, and calorie intake a combination of a Mulberry leaf extract and CissusQuadrangularis extract or plant powder.

### CROSS-REFERENCE TO RELATED APPLICATIONS

This application claims priority benefit to the provisional application 60/921,906, filed on Apr. 4, 2007, which is incorporated by reference herein in its entirety.

### FIELD OF THE INVENTION

This invention relates to methods and compositions for diabetes, obesity and metabolic syndrome control and management by administering a combination of a Mulberry leaf extract and a Cissus Quadrangularis plant powder or extract.

### BACKGROUND OF THE INVENTION

Obesity is a major contributor to morbidity and mortality in the developed countries today. People who are overweight or obese have much higher risk to develop diabetes and metabolic syndrome. Diet and exercise with resultant weight loss is an effective means in diabetes and metabolic syndrome control and management. However, the majority of patients cannot accomplish these goals, and efficacious pharmacotherapies would be ideal adjunct to diet and exercise.

Mulberry leaf extract, a traditional food and herb used in Asia, was found to possess anti-diabetic and anti-lipidemia properties in animal and human clinical studies. In vitro biochemical tests found that the herb extract was effective in inhibiting alpha glucosidase which converts complex sugars or disaccharides (such as sucrose) into absorbable single sugars.

Previous studies confirm that mulberry leaf extract is effective in reducing the absorption of sugars in human, an in vivo extension of its in vitro alpha glucosidase inhibition activity (1-2). It was also found that Mulberry leaf extract significantly reduced post-prandial or post-meal blood sugar levels in both healthy subjects and type 2 diabetics (2). Furthermore, mulberry leaf extract was found to significantly reduce the fluctuations of blood sugar levels after meal, which is thought a major contributor to the diabetic complications (2).

Cissus quadrangularis extract, a traditional India herb remedy for bone healing and ulcer curing, is reported to possess weight loss effects in two recent, double blind, placebo controlled clinical trials (9-10). See also U.S. Pat. No. 7,175,859; U.S. Pub. Nos. 2007/0048392, and 2005/0048141. In addition, Cissus quadrangularis extract has also been found to reduce blood glucose and serum lipid levels in trial subjects. It has been suggested that decreasing the absorption of lipid and carbohydrate are the possible mechanisms of Cissus Quadrangularis extract in reducing body weight. Another likely mechanism is working as an appetite suppressant, since serum 5-HT level is increased by 53% after 6 weeks' usage of Cissus Quadrangularis extract (10). 5-HT and its receptors, especially 5-HT2c, are implicated influencing in intra- and post-meal satiety and therefore are regarded as potential targets for obesity treatments (11-13).

### BRIEF SUMMARY OF THE INVENTION

The present invention provides compositions and methods for using mulberry leaf extract in combination with Cissus quadrangularis extract or Cissus quadrangularis plant powder to create a product which can 1) reduce the amount of carbohydrate absorbed; 2) reduce post-meal blood sugar elevation and blood sugar fluctuations; and 3) create sense of satiety and cut food craving and therefore be an effective agent for obesity, diabetes and metabolic syndrome control and management.

The present invention provides a composition comprising a) a mulberry leaf extract and b) a Cissus quadrangularis extract or a Cissus quadrangularis plant powder.

In some embodiments, the composition further comprises a pharmaceutically acceptable carrier. The composition may be in tablets, capsules, powders or beverages. The composition may be included in food product or beverage.

The Cissus quadrangularis extract may be an aqueous extract, an alcohol extract, or an ethyl acetate extract. The Cissus quadrangularis plant powder may be a nano-milled plant powder.

In some embodiments, the composition further comprises one or more ingredients selected from the group consisting of lipoic acid, chromium polynicotinate, chromium picolinate, insulina leaf extract, banaba leaf extract, vitamin B1, vitamin B6, folic acid, vitamin B12, green tea extract, and black tea extract. In some embodiments, the composition comprises mulberry leaf extract, green tea extract, black tea extract, Cissus quadrangularis alcoholic extract, lipoic acid, chromium picolinate, vitamin B6, and vitamin B12. In some embodiments the composition comprises mulberry leaf extract, Cissus quadrangularis alcoholic extract, lipoic acid, and chromium picolinate. In some embodiments, the composition comprises mulberry leaf extract, Cissus quadrangularis alcoholic extract, alpha lipoic acid and chromium polynicotinate. In some embodiments, the composition comprises leaf extract, Cissus quadrangularis alcoholic extract, alpha lipoic acid, chromium polynicotinate, insulina leaf extract, banaba leaf extract, vitamin B1, vitamin B6, folic acid and vitamin B12.

The invention also provides a method for controlling food, energy or calorie intake in an individual comprising administering to the individual a composition described herein. In some embodiments, the composition comprises a) a mulberry leaf extract and b) a Cissus quadrangularis extract or a Cissus quadrangularis plant powder.

In some embodiments, the composition is administered orally. In some embodiments, the composition is administered before meal or with meal. In some embodiments, the composition is administered about 15 minutes to about one hour before meal.

The invention also provides for controlling food, energy or calorie intake in an individual comprising administering to the individual a composition comprising a mulberry leaf extract in conjunction with a composition comprising a Cissus quadrangularis extract or a Cissus quadrangularis plant powder.

In some embodiments, the two compositions are administered simultaneously. In some embodiments, the two compositions are administered at different times. In some embodiments, one or both compositions are administered orally. In some embodiments, one or both compositions are administered before meal, for example, about 15 minutes to about one hour before meal. In some embodiments, one or both compositions are administered with meal.

The invention also provides a kit for use in any of the methods described herein comprising the compositions described herein. The kit may further comprise instructions for using the methods described herein.

The invention provides a kit comprising a composition comprising a) a mulberry leaf extract and b) a Cissus quadrangularis extract or a Cissus quadrangularis plant powder.

The invention also provides a kit comprising a) a composition comprising the mulberry leaf extract and b) a composition comprising the Cissus quadrangularis extract or plant powder. The kit may further comprise instructions for administration of the two compositions simultaneously or at different times. In some embodiments, the two compositions are packaged together, but may or may not be in the same container.

### DETAILED DESCRIPTION OF THE INVENTION

The present invention provides compositions and methods for controlling food or energy or calorie intake into the body of an individual and thus causes weight loss in that individual.

### A. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “treatment” is an approach for obtaining beneficial or desired clinical results. For purposes of this invention, beneficial or desired clinical results include, but are not limited to, one or more of the following: improvement or alleviation of any aspect of reducing blood glucose level or maintaining healthy blood glucose level, and reducing body weight or maintaining body weight within the normal range.

An “effective amount” is an amount sufficient to effect beneficial or desired clinical results including controlling food or energy or calorie intake. An effective amount, in the context of this invention, may also be amounts of using two agents described herein such that a synergy is achieved. An “effective amount” of two agents described herein can result in a synergistic effect as compared to administering each agent alone.

An “individual” is a mammal, more preferably a human. Mammals also include, but are not limited to, farm animals, sport animals, pets, primates, horses, cows, dogs, cats, mice and rats.

As used herein, administration “in conjunction” includes simultaneous administration and/or administration at different times. As used herein, administration in conjunction is meant to encompass any circumstance wherein the two agents described herein are administered to an individual, which can occur simultaneously and/or separately. As further discussed herein, it is understood that the two agents can be administered at different dosing frequencies or intervals. It is understood that the two agents can be administered using the same route of administration or different routes of administration.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “consisting” and/or “consisting essentially of” aspects and embodiments.

### B. Compositions

The mulberry leaf extract and Cissus Quadrangularis extract or plant powder may be formulated in the same composition or in different compositions. One or more of other ingredients may also be included in the composition. For example, lipoic acid, chromium polynicotinate, chromium picolinate, insulina leaf extract, banaba leaf extract, vitamin B1, vitamin B6, folic acid, vitamin B12, green tea extract, and black tea extract may be included in the composition. In some embodiments, the Cissus Quadrangularis powder is nano-milled (15). In some embodiments, the compositions described herein further comprise a pharmaceutically acceptable excipient or carrier. In some embodiments, the compositions is in tablets, capsules, powders or beverages. The compositions may be included in food product or beverage. In some embodiments, the composition is for use in any of the methods described herein (such as methods for treating diabetes and/or obesity).

The compositions provided in the present invention can further comprise pharmaceutically acceptable carriers, excipients, or stabilizers (Remington: The Science and Practice of Pharmacy 20th Ed. (2000) Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### C. Methods of Administration

The invention also provides a method for controlling food, energy or calorie intake in an individual comprising administering to the individual a composition described herein. In some embodiments, the composition comprises a) a mulberry leaf extract and b) a Cissus quadrangularis extract or a Cissus quadrangularis plant powder.

The invention also provides a method for reducing blood sugar level in an individual having diabetes comprising administering to the individual a composition described in herein.

The invention also provides a method for reducing body weight in an individual comprising administering to the individual a composition described herein.

In some embodiments, the composition is administered orally. In some embodiments, the composition is administered before meal or with meal. In some embodiments, the composition is administered (e.g., taken orally) about 15 minutes to about 2 hours before meal; for example, about 15 minutes, about 30 minutes, about 1 hour, about 1.5 hours, or about 2 hours before meal.

The invention also provides for controlling food, energy or calorie intake in an individual comprising administering to the individual a composition comprising a mulberry leaf extract in conjunction with a composition comprising a Cissus quadrangularis extract or a Cissus quadrangularis plant powder.

In some embodiments, the two compositions are administered simultaneously. In some embodiments, the two compositions are administered at different times. In some embodiments, one or both compositions are administered orally. In some embodiments, one or both compositions are administered before meal. In some embodiments, one or both compositions are administered (e.g., taken orally) about 15 minutes to about 2 hours before meal; for example, about 15 minutes, about 30 minutes, about 1 hour, about 1.5 hours, or about 2 hours before meal. In some embodiments, one or both compositions are administered with meal.

The methods of the present invention may be used for treating or preventing diabetes, metabolic syndrome, obesity, or overweight. The compositions described herein may be administered to an individual having diabetes or being at risk of diabetes, or to an individual being obese or at risk of obesity.

Administration methods and dosage are further described in Exemplary Embodiments and Examples. Various administration methods and dosage described herein may be used.

### D. Kits

The invention also provides a kit comprising the compositions described herein. The kit may further comprise instructions for using the methods described herein. The compositions described herein may be in a container with a label. Suitable containers include, for example, bottles and vials. The label may contain instructions for using the methods described herein.

### E. Exemplary Embodiments

Mulberry leaf and Cissus quadrangularis extracts have different functions and different mechanisms. Mulberry leaf extract reduces post-meal blood sugar level and reduces the absorption of carbohydrates . Alcoholic Cissus extract reduces appetite and potentially for weight control, and has no or not strong effect on post-meal blood sugar level. The combination of Mulberry leaf extract and Cissus Quadrangularis may act synergistically and has a strong effect in diminishing food or energy or calorie intake into the body, and thus have strong effects in lowering body weight.

Description of Products:

1. Mulberry Leaf Extract:

Water or alcohol extract of Mulberry leaves, further concentrated by chemical extraction and/or column chromatography, have 50-100% (more specifically 80-90%) inhibition on alpha glucosidase, based on an in vitro alpha glucosidase inhibition assay (14). Mulberry leaf extract is commercially available and can be purchased from NatureGen, Inc. (San Diego, Calif.).

2. Cissus Quadrangularis Extract

a. Alcohol extract of Cissus Quadrangularis (leaves and stems)

Cissus Quadrangularis (such as leaves and stems) extract may be obtained by extracting with alcohol/water solvent. For example, about 50% to about 95% alcohol in water may be used. In an example, Cissus Quadrangularis (leaves and stems) are boiled in 3 times volume 50% alcohol for 4 hours. The extract solution is collected and dried under vacuum or freeze drying to get the extract powder. Alcoholic extract of Cissus Quadrangularis is also commercially available.

b. Ethyl acetate extract of Cissus Quadrangularis (leaves and stems)

Cissus Quadrangularis (such as leaves and stems) extract may be obtained by extracting with ethyl acetate/water solvent. For example, about 60% to about 100% ethyl acetate in water may be used. In an example, Cissus Quadrangularis (leaves and stems) are boiled in 3 times volume 60% ethyl acetate solution (a mixture of ethyl acetate and water) for four hours. The extract solution is collected and dried under vacuum or freeze drying to get the extract powder.

c. Aqueous extract of Cissus Quadrangularis

Cissus Quadrangularis (such as leaves and stems) extract may be obtained by extracting with water. For example, leaves, stems and/or roots may be boiled in water for at least 1 hour. The extract solution is collected and dried under vacuum or freeze drying to get the extract power.

### 3. Cissus Quadrangularis Powder

a. Regular Cissus Quadrangularis powder (mesh size 10-200)

Cissus Quadrangularis powder is commercially available or can be prepared using any known methods. In an example, dry Cissus Quadrangularis (leaves and stems) are first cut into small pieces (about 1 cm³). The small Cissus Quadrangularis pieces are then processed by a high power grinder to make powders.

b. Nano-milled Cissus Quadrangularis powder (mesh size 400-1000)

Nano-milled Cissus Quadrangularis may be produced using known methods. For example, the above described regular Cissus Quadrangularis powder (mesh size 10-200) may be further processed by a nano-milled machine and to make nano-milled Cissus Quadrangularis (400-1000).

Product Administration:

The intended usage of the combinations, either in one composition or in two compositions, is two to three times a day, before or with a meal. Each serving should comprise the following: Mulberry extract from about 50 mg to about 1500 mg (which has 60-90% inhibition of alpha Glucosidase), for example from about 250 mg to about 1000 mg (e.g., about 600 mg); and Cissus quadrangularis: a) regular powder, from about 100 mg to about 1000 mg, for example from about 200 mg to about 750 mg or about 500 mg to about 750 mg; b) nano-milled powder, from about 100 mg to about 900 mg, for example about 200 to about 500 mg; or c) Extract, from about 50 mg to about 500 mg, for example about 100 mg to about 300 mg.

Dosage Form:

The combinations can be in tablets and/or capsules; powders; beverage; or food (such as pizza or pasta or bar, ingredients)

Examples of Use:

1. Tablets and Capsules:

In order to reduce body weight, one subject takes 2-4 tablets or capsules with water before each meal. Each capsule or tablet contains about 300 mg Mulberry extract and about 100 mg Cissus Quadrangularis extract with other inactive excipients.

2. Powders

In order to reduce body weight, prior to meal, one subject mixes water with one spoon of blended powder which consists of about 600 mg Mulberry extract and about 300 mg nano-milled Cissus quadrangularis powder with other inactive excipients including flavor additives such as lemon, orange or banana. The subject then drinks the mixture.

3. Beverage

In order to control blood glucose and/or body weight, prior to meal, one subject drinks a 8-fluid ounce can or bottle which contains about 500 mg Mulberry extract and about 500 mg regular Cissus quadrangularis powder with other inactive excipients including flavor additives such as lemon, orange or banana.

4. Low Glycemic Index Food, such as Pasta, Bread, Pizza or Bar

Food, such as pasta, bread, pizza, bar and etc, can be made with the addition of a Mulberry leaf extract and Cissus Quadrangularis or a derivative thereof. This kind of food can be low glycemic index food, because much less glucose is going to be produced and absorbed by the body in comparing with other food without the addition of these ingredients.

5. Pre-Mixed Powders

A Mulberry leaf extract and Cissus quadrangularis extract (all in the powder form) can be mixed or blended in a specific ratio, such as Mulberry extract:Cissus Quadrangularis extract ratio is 500:200 to produce a premixed powder. Such premixed powder can be used to make capsules, tablets, beverages and/or used as food ingredients.

6. Formulated with Other Ingredients

Other ingredients can be added to the mulberry leaf extract and Cissus quadrangularis combination to further enhance the efficacy. Two examples are given at below:

Formula A:

Mulberry leaf extract

Green Tea Extract

Black Tea Extract

Cissus Quadrangularis alcoholic extract

Lipoic acid

Chromium Picolinate

Vitamin B6

Vitamin B12

Formula B:

Mulberry leaf extract

Cissus Quadrangularis alcoholic extract

Lipoic acid

Chromium Picolinate

### EXAMPLES

Testing of various extractions of Cissus Quadrangularis indicated that alcohol extracted Cissus Quadrangularis had an appetite suppressing effect which was stronger than that of the water extracted Cissus Quadrangularis. Therefore, alcohol extracted Cissus Quadrangularis was included in Formula C and Formula D and to help suppressing appetite and reducing food intake. It was also found that the appetite suppression effect was stronger if the product was taken 1 hour before food.

### Test of Formula C on Post-Meal Blood Sugar Levels

Formula C which contains the following ingredients:
Mulberry leaf extract: 600 mg (75% alcohol extract; commercially available from NatureGen, Inc.)
Cissus Quadrangularis alcoholic extract: 180 mg (50% alcohol extract; commercially available from NatureGen, Inc.)
Alpha lipoic acid: 200 mg (synthesized)
Chromium Polynicotinate: 200 mcg (synthesized)

Formula C which contains 980.2 mg of ingredients in total, was put into two capsules.

Effect of Formula C on post-meal blood sugar levels was tested. On test day one, 5 human subjects were instructed to drink 50 grams of sucrose (table sugar) dissolved in 1 large glass of water and to eat 3 pieces of white bread for breakfast after overnight fasting. Blood sugar levels were measured by blood glucose meter from finger tip blood samples at 0, 15, 30 and 60 minutes after eating. On test day two, same procedures as day one were followed, except each subject took formula C (contained in two single O capsules) 15 minutes before taking the food. It was found that on average, the peak post-meal blood sugar levels were reduced by 41% in the 5 tested subjects.

### Test of Formula D on Post-Meal Blood Sugar Levels and Weight

Formula D contains the following ingredients:

Mulberry leaf extract: 600 mg (75% alcohol extract; commercially available from NatureGen, Inc.)

Cissus Quadrangularis alcoholic extract: 180 mg (50% alcohol extract; commercially available from NatureGen, Inc.)

Alpha Lipoic acid: 200 mg (synthesized; commercially available from US nutraceutical ingredient suppliers)

Chromium Polynicotinate: 200 mcg (synthesized; commercially available from US nutraceutical ingredient suppliers)

Insulina (Cissus Sicyoides) Leaf Extract: 6 mg (water extract; commercially available from Brazil herbal extractors)

Banaba (Lagerstroemia Speciosal) Leaf Extract (18% corosolic acid): 6 mg (alcohol water extract; commercially available from US nutraceutical ingredient suppliers)

Vitamin B1 (as Thiamine Hydrochloride): 4 mg (synthesized; commercially available from US nutraceutical ingredient suppliers)

Vitamin B6 (as Pyridoxine Hydrochloride): 4 mg (synthesized; commercially available from US nutraceutical ingredient suppliers)

Folic Acid: 400 mcg (synthesized; commercially available from US nutraceutical ingredient suppliers)

Vitamin B12 (as Cyanocobalamin): 15 mcg (synthesized; commercially available from US nutraceutical ingredient suppliers)

Formula D, which contains 1000.615 mg of ingredients in total, was put into two capsules.

Effect of Formula D on post-meal blood sugar levels was tested. On test day one, 6 human subjects were instructed to drink 50 grams of sucrose (table sugar) dissolved in 1 large glass of water and to eat 3 pieces of white bread for breakfast after overnight fasting. Blood sugar levels were measured by blood glucose meter from finger tip blood samples at 0, 15, 30 and 60 minutes after eating. On test day two, same procedures as day one were followed, except each subject took formula D (contained in two single O capsules) 15 minutes before taking the food. It was found that on average, the peak post-meal blood sugar levels were reduced by 41% in the 6 tested subjects.

Effect of Formula D on weight was tested. Five people were taking Formula D (in two single O capsules), twice a day, 15 minutes to 60 minutes before lunch and dinner every day for 4 months. Body weights were measured in mornings before food, with minimal clothing. Following is the net change of body weight in each tested subject after 4 months:
Subject 1: lost 14 pounds
Subject 2: lost 11 pounds
Subject 3: lost 9 pounds
Subject 4: lost 5 pounds
Subject 5: lost 4 pounds

This suggests that Formula D was effective on weigh loss.

Although the foregoing invention has been described in some detail by way of illustration and example for purposes of clarity of understanding, the descriptions and examples should not be construed as limiting the scope of the invention.

### REFERENCES

1. Mudra M, Ercan-Fang N, Zhong L, Fume J and Levitt M. Influence of mulberry leaf extract on the blood glucose and breath hydrogen response to ingestion of 75 g of sucrose by subjects with type 2 diabetes and controls. Diabetes Care 2007; (In press)
2. Hansawasdi C and Kawabata J. Alpha-glucosidase inhibitory effect of mulberry (Moms alba) leaves on Caco-2. Fitoterapia 2006; 77:568-73
3. Enc F Y, Imeryuz N, Akin L, Turoglu T, Dede F, Haklar G, Tekesin N, Bekiroglu N, Yegen B C, Rehfeld J F, Holst J J and Ulusoy N B. Inhibition of gastric emptying by acarbose is correlated with GLP-1 response and accompanied by CCK release. Am J Physiol Gastrointest Liver Physiol 2001; 281:G752-63
4. Göke B and Herrmann-Rinke C. The evolving role of α-glucosidase inhibitors. Diabetes/Metabolism Reviews 1998; 14:S31-8
5. Nystrom T, Gutniak M K, Zhang Q, Zhang F, Holst J J, Ahren B and Sjoholm A. Effects of glucagon-like peptide-1 on endothelial function in type 2 diabetes patients with stable coronary artery disease. Am J Physiol Endocrinol Metab 2004; 287:E1209-15
6. Toft-Nielsen M B, Madsbad S and Holst J J. Continuous subcutaneous infusion of glucagon-like peptide 1 lowers plasma glucose and reduces appetite in type 2 diabetic patients. Diabetes Care 1999; 22:1137-43
7. Verdich C, Toubro S, Buemann B, Lysgard Madsen J, Juul Holst J and Astrup A. The role of postprandial releases of insulin and incretin hormones in meal-induced satiety—effect of obesity and weight reduction. Int J Obes Relat Metab Disord 2001; 25:1206-14
8. Gutzwiller J P, Degen L, Heuss L and Beglinger C. Glucagon-like peptide 1 (GLP-1) and eating. Physiol Behav 2004; 82:17-9
9. The use of a Cissus quadrangularis formulation in the management of weight loss and metabolic syndrome. Lipids Health Dis 2006; 5:24.

10. Oben J E, Enyegue D M, Fomekong G I, Soukontoua Y B and Agbor G A. The effect of Cissus quadrangularis (CQR-300) and a Cissus formulation (CORE) on obesity and obesity-induced oxidative stress. Lipids Health Dis 2007;6:4
11. Smith B K, York D A and Bray G A. Activation of hypothalamic serotonin receptors reduced intake of dietary fat and protein but not carbohydrate. Am J Physiol 1999; 277:R802-11
12. Schuhler S, Clark A, Joseph W, Patel A, Lehnen K, Stratford E, Horan T L, Fone K C F and Ebling F J P. Involvement of 5-HT2C recptors in the regulation of food intake in Siberian Hamsters. J Neuroendo 2005; 17:276-85
13. DeWald T, Khaodhiar L, Konahue M P and Blackburn G. Pharmacological and surgical treatments for obesity. Am Heart J 2006; 151:604-24
14. Asano N, Oseki K, Tomioka E, Kizu H, and Matsui K. N-containing sugars from Morus alba and their glycosidase inhibitory activities. Carbohydr. Res. 1994; 259:243-55.
15. Liu, Z. Dissolution study of active ingredients in nanomilled Angelica sinensis. China Annual Pharmaceutical Conference Abstract: 2001:911-914.
16. Jainu M, Vijai Mohan K, Shyamala Devi C S. Gastroprotective effect of Cissus quadrangularis extract in rats with experimentally induced ulcer. Indian J Med Res. 2006 June;123(6):799-806.
17. Jainu M, Devi C S. Gastroprotective action of Cissus quadrangularis extract against NSAID induced gastric ulcer: role of proinflammatory cytokines and oxidative damage. Chem Biol Interact. 2006 Jul. 10;161(3):262-70. Epub 2006 May 1.
18. Støa-Birketvedt G. Effect of cimetidine suspension on appetite and weight in overweight subjects. BMJ. 1993 Apr. 24;306(6885):1091-3.
19. Støa-Birketvedt G, Paus P N, Ganss R, Ingebretsen O C, Florholmen J. Cimetidine reduces weight and improves metabolic control in overweight patients with type 2 diabetes. Int J Obes Relat Metab Disord. 1998 November;22(11):1041-5.
20. Støa-Birketvedt G, Løvhaug N, Vonen B, Florholmen J. H2-receptor antagonist reduces food intake and weight gain in rats by non-gastric acid secretory mechanisms. Acta Physiol Scand. 1997 December;161(4):489-94.
21. Fox M T, Gerrelli D, Shivalkar P, Jacobs D E. Effect of omeprazole treatment on feed intake and blood gastrin and pepsinogen levels in the calf Res Vet Sci. 1989 March;46(2):280-2.
22. Oben J, Kuate D, Agbor G, Momo C, Talla X. The use of a Cissus quadrangularis formulation in the management of weight loss and metabolic syndrome. Lipids Health Dis. 2006 Sep. 2;5:24.

The claimed invention is:

1. A composition comprising: a) a mulberry leaf extract; and b) a Cissus quadrangularis extract or a Cissus quadrangularis plant powder.

2. The composition of claim 1, wherein the Cissus quadrangularis powder is a nano-milled plant powder.

3. The composition of claim 1, wherein the Cissus quadrangularis extract is an aqueous extract, an alcohol extract, or an ethyl acetate extract.

4. The composition of claim 1, further comprising one or more ingredients selected from the group consisting of lipoic acid, chromium polynicotinate, chromium picolinate, insulina leaf extract, banaba leaf extract, vitamin B1, vitamin B6, folic acid, vitamin B12, green tea extract, and black tea extract.

5. The composition of claim 1, further comprising alpha lipoic acid and chromium polynicotinate.

6. The composition of claim 1, further comprising alpha lipoic acid, chromium polynicotinate, insulina leaf extract, banaba leaf extract, vitamin B1, vitamin B6, folic acid, and vitamin B12.

7. A method for controlling food, energy or calorie intake in an individual comprising administering to the individual the composition of claim 1.

8. The method of claim 7, wherein the composition is administered orally.

9. The method of claim 7, wherein the composition is administered before meal.

10. The method of claim 9, wherein the composition is administered about 15 minutes to about one hour before meal.

11. The method of claim 7, wherein the composition is administered with meal.

12. The method of claim 7, wherein the individual has diabetes or is at risk of diabetes.

13. The method of claim 7, wherein the individual is obese or is at risk of obesity.

14. A method for controlling food, energy or calorie intake in an individual comprising administering to the individual a composition a mulberry leaf extract in conjunction with a composition comprising a Cissus quadrangularis extract or a Cissus quadrangularis plant powder.

15. The method of claim 14, wherein the two compositions are administered simultaneously.

16. The method of claim 14, wherein the two compositions are administered at different times.

17. The method of claim 14, wherein one or more of the two compositions are administered orally.

18. The method of claim 14, wherein one or more of the two compositions are administered before meal.

19. The method of claim 14, wherein one or more of the two compositions are administered with meal.

20. The method of claim 18, wherein the two compositions are administered about 15 minutes to about one hour before meal.

21. The method of claim 14, wherein the individual has diabetes or is at risk of diabetes.

22. The method of claim 14, wherein the individual is obese or is at risk of obesity.

23. A kit comprising a composition comprising a) a mulberry leaf extract; and b) a Cissus quadrangularis extract or a Cissus quadrangularis plant powder.

24. The kit of claim 23, further comprises an instruction for using the composition for controlling food, energy and calorie intake in an individual.

25. A kit comprising a) a composition comprising the mulberry leaf extract and b) a composition comprising the Cissus quadrangularis extract or plant powder.

26. The kit of claim 25, further comprises an instruction for using the composition for controlling food, energy and calorie intake in an individual.
